(19) United States
(12) Patent Application Publication
(10) Pub. No.: US 2020/0038239 A1
(43) Pub. Date: Feb. 6, 2020

(54) NONLINEAR COLLAGEN CROSSLINKING USING A SINGLE, AMPLIFIED, FEMTOSECOND LASER PULSE

(71) Applicant: The Regents of the University of California, Oakland, CA (US)

(72) Inventors: Eric Mikula, Irvine, CA (US); Samantha Bradford, IRVINE, CA (US); Donald J. Brown, Santa Clarita, CA (US); Tibor Juhsa, Corona del Mar, CA (US); James V. Jester, IRVINE, CA (US)

(21) Appl. No.: 16/482,220

(22) PCT Filed: Jan. 30, 2018
(86) PCT No.: PCT/US18/16005

§ 371 Date: Jul. 30, 2019

Related U.S. Application Data
(60) Provisional application No. 62/452,671, filed on Jan. 31, 2017.

(51) Int. Cl. A61F 9/008; A61B 18/20

(52) U.S. Cl. CPC A61F 9/008; A61B 18/203; A61F 2009/00895; A61F 2009/00872; A61B 2018/20353

(57) ABSTRACT

A laser beam delivery system including an amplified femtosecond (FS) laser device coupled to a nonlinear optical parametric amplifier (NOPA) configured to select a FS laser wavelength and to amplify input amplified femtosecond (FS) laser pulses of from 700 to 2500 nm to generate a single, parametrically amplified output FS pulse having a pulse energy of from 0.1-100 μJ, wherein the NOPA uses an average power of below 46.1 mW to amplify the input FS laser pulses. Also disclosed is a method of nonlinear optical photodynamic irradiation of a target.

### STATEMENT REGARDING FEDERALLY SPONSORED R&D

This invention was made with government support under EY24600, EY007348 and EY018655, awarded by The National Eye Institute of the National Institutes of Health. The government has certain rights in the invention.

### BACKGROUND

### Field

The disclosure relates to the field of using nonlinear optical photodynamic therapy (NLO-PDT) to cause collagen crosslinking using infrared light and riboflavin in tissues, such as the eye.

### Description of the Related Art

Overall, refractive errors are the most common vision-related disorder affecting over 200 million Americans, for which Americans pay over $16 billion annually for treatment including glasses, contact lenses and refractive surgery (Wittenborn, J. and Rein, D. 2013. Cost of Vision Problems: The economic burden of vision loss and eye disorders in the United States, Presented to Prevent Blindness America). Of these patients, over 60 million people suffer from low degrees of myopia (<−2 diopters), another 60 million suffer from astigmatism (<2 diopters) (Vitale, S. et al. 2008 Arch Ophthalmol 126: 1111-1119), while an additional 20 million individuals have low degrees of presbyopia requiring <2 diopters addition correction (Lindstrom, R. L et al. 2013 Curr Opin Ophthalmol 24: 281-287). Potentially, there are over 140 million patients with refractive errors that are mostly treated with glasses and contact lenses. It is widely recognized that no single method for correcting refractive errors is either appropriate for or appealing to all patients (Riley, C. and Chalmers, R. L., 2005 Optom Vis Sci 82: 555-561), indicating that a novel, non-invasive laser strategy may have the potential to disrupt the current practice for treating these patients. Specifically, nonlinear optical crosslinking (NLO CXL) is a non-invasive technology, unlike LASIK surgery, in that by using femtosecond (FS) laser light alone, along with a photo-initiator, regional stiffening can be achieved that will induce a change in the corneal tissue mechanics that control corneal shape and refractive power between 0.5 and 2 diopters. Over the past 10 years, a yearly average of 1 million LASIK surgeries have been performed on a potential market of 60 million individuals with greater than −2 diopters of myopia. This indicates that LASIK surgery has been able to capture about 1.6% of the myopia market. Extrapolating from the LASIK experience, penetration of NLO CXL to 1.0% of the potential market would result in capturing 1.4 million refractive error patients/year. Assuming a fee charge of $100 for each procedure/eye, under the $150 charge for using the LASIK surgery device, and presuming that each individual has two procedures, both left and right eye, the NLO CXL refractive product has the potential to generate $280 Million in yearly revenue in the US alone. Assuming world-wide acceptance and distribution, this number can easily grow above $1 Billion.

Numerous reports in the scientific literature relate to corneal collagen crosslinking, but none relate to the use of femtosecond lasers to activate photosensitizers in the cornea. Several recent papers report evaluation of collagen crosslinking following femtosecond laser generated tunnels in the cornea, but the researchers did not use the laser to activate a photosensitizer. In the past, crosslinking in the cornea has used UV light to activate the photosensitizer, riboflavin. The disadvantage of this approach is that it uses nonfocused light, which broadly and nonspecifically generates free radicals throughout the tissue volume, wherever the light penetrates.

### SUMMARY

We disclose the use of NLO CXL to perform refractive corrections of low degrees of myopia, hyperopia, presbyopia and astigmatism less than 2 diopters. NLO CXL can be performed using regeneratively amplified FS laser pulses from 5 kHz to 50 kHz. Cross linking can be performed with pulse energies between 0.1 to 100 μJ. Cross linking can be performed with energy densities of 1-100 J/cm². Effective cross linking can be achieved using an average power of less than 46.1 mW, below the ANSI thermal poser limits for laser exposure to the eye.

Some embodiments relate to a laser beam delivery system including an amplified femtosecond (FS) laser device coupled to a nonlinear optical parametric amplifier (NOPA) configured to select a FS laser wavelength and to amplify input amplified femtosecond (FS) laser pulses of from 700 to 2500 nm to generate a single, parametrically amplified output FS pulse having a pulse energy of from 0.1-100 p, wherein the NOPA uses an average power of below 46.1 mW to amplify the input FS laser pulses.

In some embodiments, the amplified FS laser device is configured to provide a repetition rate of 5 kHz to 50 kHz pulses.

In some embodiments, the NOPA is configured to parametrically amplify 760 nm pulses.

In some embodiments, the system is configured to focus 760 nm light with a variable 0.1-0.3 numerical aperture (NA) objective.

In some embodiments, the NOPA is configured to provide a single output pulse of about 2 μJ pulse energy having an average power of about 12 mW or less.

In some embodiments, the system includes a tracker that automatically monitors position of a subject or a tissue so that the device is able to compensate for movement of the subject or tissue.

Some embodiments relate to a method of nonlinear optical photodynamic irradiation of a target, the method including exposing the target to a single amplified femtosecond laser pulse, wherein the amplified femtosecond laser pulse has a wavelength of from about 700 nm to 2500 nm, and wherein the single amplified femtosecond laser pulse has a pulse energy of from 0.1-100 μJ and an average power of less than 46.1 mW.

In some embodiments of the method, the single amplified femtosecond laser pulse is applied at an energy density below 100 J/cm².

In some embodiments of the method, the single pulse is for a duration of about 150 femtoseconds.

In some embodiments of the method, the method includes using regeneratively amplified pulses from 5 kHz to 50 kHz.

In some embodiments of the method, the method includes using pulse energies of between 0.1-100 μJ.

In some embodiments of the method, the method includes using energy densities of 1-100 J/cm2.

In some embodiments of the method, the method includes pretreating the target with a photosensitive agent which is capable of generating free radicals within the treatment volume upon irradiation.

In some embodiments, the photosensitive agent comprises riboflavin.

Some embodiments relate to a method of nonlinear optical photodynamic therapy of a tissue, the method including exposing the tissue to a single amplified femtosecond laser pulse, wherein the amplified femtosecond laser pulse has a wavelength of from about 700 nm to 2500 nm to minimize cellular damage by reducing energy level of the laser light and increasing its depth of penetration into the tissue, wherein the single amplified femtosecond laser pulse has a pulse energy of from 0.1-100 μJ and an average power of less than 46.1 mW.

In some embodiments of the method, the single amplified femtosecond laser pulse is applied at an energy density below 100 J/cm².

In some embodiments of the method, the single pulse is for a duration of about 150 femtoseconds.

In some embodiments of the method, the method includes using regeneratively amplified pulses from 5 kHz to 50 kHz.

In some embodiments of the method, the method includes using pulse energies of between 0.1-100 μJ.

In some embodiments of the method, the method includes using energy densities of 1-100 J/cm2.

In some embodiments of the method, the tissue is a cornea.

In some embodiments of the method, the method includes applying specific geometric patterns of collagen crosslinking (CXL) to induce defined and controllable corneal stiffening.

In some embodiments of the method, the method includes producing 2 diopters or less of corneal flattening and/or steepening.

In some embodiments of the method, refractive correction of low degrees of myopia, hyperopia, presbyopia and astigmatism is achieved.

In some embodiments of the method, the method includes pretreating the tissue with a photosensitive agent which is capable of generating free radicals within the treatment volume upon irradiation.

In some embodiments, the photosensitive agent comprises riboflavin.

In some embodiments, the pulsed infrared laser light within the tissue provides sufficient intensity and length of irradiation to cause collagen crosslinking (CXL).

In some embodiments, the pulsed infrared laser light within the tissue provides sufficient intensity and length of irradiation to effectively provide anti-microbial mediation.

In some embodiments, the illustrated embodiments of the invention are directed to apparatus and methods of using nonlinear optical (NLO), femtosecond-near infrared lasers used to activate photosensitizing chemicals in the cornea for various corneal treatments including corneal stiffening to treat corneal ectasia, refractive errors and astigmatism as well as provide antimicrobial and tumorcidal effects.

Some of the illustrated embodiments are directed to a method of nonlinear optical photodynamic therapy of tissue including the steps of providing pulsed infrared laser light for multiphoton tissue exposure, and selectively focusing the pulsed infrared laser light within the tissue at a focal volume to activate a photosensitizing agent to generate free radicals within a highly resolved axial and lateral spatial domain in the tissue.

The method may further include the step of pretreating the tissue with the photosensitive agent prior to selectively focusing the pulsed infrared laser light within the tissue. The photosensitive agent includes but not limited to riboflavin.

The step of providing pulsed infrared laser light includes providing near-infrared light to minimize cellular damage by reducing photon energy level of the laser light and increasing depth penetration into the tissue.

In embodiments where the tissue is a cornea the step of selectively focusing the pulsed infrared laser light within the tissue includes providing sufficient intensity and length of irradiation to cause collagen crosslinking (CXL) effective for corneal stiffening.

In embodiments where the tissue is a cornea the step of selectively focusing the pulsed infrared laser light within the tissue includes providing sufficient intensity and length of irradiation to effectively provide anti-microbial mediation to treat a corneal infection.

In embodiments where the tissue is a cornea the step of selectively focusing the pulsed infrared laser light within the tissue includes providing sufficient intensity and length of irradiation to effectively inhibit corneal swelling in bullous keratopathy.

In embodiments where the tissue is a cornea the step of selectively focusing the pulsed infrared laser light within the tissue includes providing sufficient intensity and length of irradiation to effectively kill cells, bacteria, tumors or neovascular vessels growing into the avascular cornea.

In some embodiments, the step of selectively focusing the pulsed infrared laser light within the tissue includes providing sufficient intensity and length of irradiation to effectively activate the photosensitizing agent only at the focal volume.

In some embodiments where the tissue is a cornea the step of selectively focusing the pulsed infrared laser light within the tissue includes providing sufficient intensity and length of irradiation to effectively cause corneal stiffening by collagen crosslinking to precisely stiffen weakened corneas, including keratoconus and post-LASIK ectasia.

In some embodiments where the tissue is a cornea the step of selectively focusing the pulsed infrared laser light within the tissue includes providing sufficient intensity and length of irradiation to effectively cause corneal stiffening, flattening and steepening to precisely stiffen, flatten and steepen regions of the cornea to treat astigmatism and refractive errors associated with myopia, hyperopia and presbyopia.

In some embodiments where the tissue is a cornea the step of selectively focusing the pulsed infrared laser light within the tissue includes providing sufficient intensity and length of irradiation to effectively treat bacterial, fungal, and amoebic infections of the eye without antibiotics.

In some embodiments where the tissue is a cornea the step of selectively focusing the pulsed infrared laser light within the tissue includes providing sufficient intensity and length of irradiation to effectively kill labeled tumor cells in the eye following loading with photosensitizing dyes.

In some embodiments where the tissue is a cornea the step of selectively focusing the pulsed infrared laser light within the tissue includes providing sufficient intensity and length of irradiation to effectively treat clinical diseases including keratoconus, post-LASIK ectasia, astigmatism, myopia, hyperopia, infection and ocular tumors.

Some embodiments of the invention also include an apparatus for performing nonlinear optical photodynamic therapy of tissue including a pulsed infrared laser for providing multiphoton tissue exposure, a scanner for selectively and controllably moving the tissue and the beam relative to each other, and optics for selectively focusing the pulsed infrared laser light within the tissue at a point in a focal volume to activate a photosensitizing agent to generate free radicals within a highly resolved axial and lateral spatial domain in the tissue.

The pulsed infrared laser light includes a near-infrared laser to minimize cellular damage by reducing energy level of the laser light and increasing depth penetration into the tissue.

In some embodiments where the tissue is a cornea the pulsed infrared laser is arranged and configured with the optics to provide sufficient intensity and length of irradiation to cause collagen crosslinking (CXL) effective for corneal stiffening, selective activation of anti-microbial medication to treat a corneal infection, inhibition of corneal swelling in bullous keratopathy, or selective killing of cells, bacteria, tumors or neovascular vessels growing into the avascular cornea.

In some embodiments where the tissue is a cornea the selectively focused pulsed infrared laser is arranged and configured with the optics to provide sufficient intensity and length of irradiation to effectively cause corneal stiffening by collagen crosslinking to precisely stiffen weakened corneas, including keratoconus and post-LASIK ectasia.

In some embodiments where the tissue is a cornea the selectively focused pulsed infrared laser is arranged and configured with the optics to provide sufficient intensity and length of irradiation to effectively cause corneal stiffening and flattening to precisely stiffen and flatten regions of the cornea to treat astigmatism and refractive errors associated with myopia, hyperopia and presbyopia.

In some embodiments where the tissue is a cornea the selectively focused pulsed infrared laser is arranged and configured with the optics to provide sufficient intensity and length of irradiation to effectively treat bacterial, fungal, and amoebic infections of the eye without antibiotics, or to effectively kill labeled tumor cells in the eye following loading with photosensitizing dyes.

Other embodiments relate to a method of nonlinear optical photodynamic therapy of tissue including the steps of providing a focal spot of a pulsed infrared laser light for multiphoton tissue exposure through a focusing lens. The focal spot has a volume and the focusing lens has an effective numerical aperture. The focal spot is selectively, repetitively and three dimensionally positioned in the tissue in a selected volume of the tissue, which is larger than the volume of the focal spot, to expose the selected volume of tissue to the pulsed infrared laser light within a predetermined clinical time span. The focal spot is provided with a selected focal volume and predetermined safe intensity sufficient to activate a photosensitizing agent in the tissue in the volume of tissue to generate free radicals within a highly resolved axial and lateral spatial domain in the tissue by utilizing the predetermined safe intensity of the focal spot and by adjusting the volume of the focal spot of the pulsed infrared laser light by variably adjusting the effective numerical aperture of the focusing lens.

Other embodiments are characterized as an apparatus for performing nonlinear optical photodynamic therapy of tissue including a pulsed infrared laser for providing a beam for multiphoton tissue exposure having a beam width at a predetermined safe intensity. The beam position is controlled by a scanner, which selectively and controllably moves the beam relative to the tissue in an x and y plane. The scanned beam is modified by a variable beam expander for selectively varying the beam width or diameter. A focusing lens focuses the beam at a depth in the tissue with a selected focal volume and is selectively movable relative to the tissue along a z axis perpendicular to the x and y plane in order to selectively position the depth of the beam in the tissue. Adjustment of the beam expander selectively adjusts the effective numerical aperture of the focusing lens and hence the focal volume of the beam in the tissue. The focusing lens selectively focuses the pulsed infrared laser light within the tissue at a point in a focal volume to activate a photosensitizing agent to generate free radicals within a highly resolved axial and lateral spatial domain in the tissue.

### BRIEF DESCRIPTION OF THE DRAWINGS

FIG. 1 is a schematic diagram of an apparatus in which the invention may be practiced or embodied.

FIG. 2. (A) is a side cross-sectional view of a microphotograph of an NLO treated rabbit cornea. (B) is a comparative graph of the autofluorescence of a UVA and an NLO treated rabbit cornea.

FIG. 3. (A) and (B) are a side cross-sectional view and a cutaway perspective view respectively of a jig where in the elasticity of gels subject to the method of the invention are measured.

FIG. 4. (A), (B), and (C) are diagrammatic depictions of the apparatus and the scanning pattern by which the gels are irradiated using nonlinear optical photodynamics with a pulsed infrared laser light for two-photon excited fluorescence.

FIG. 5 is a data scan of a gel using second harmonic generation to determine its thickness.

FIG. 6 is a graph of the indenting force verses the indenting depth for the gels before and after irradiation according to the methodology of the invention.

FIG. 7 is a graph of the elastic modulus of the gels comprised of a control group, a UVA exposed gel, a low power (100 mW) nonlinear optic (NLO) exposed gel and a high power (150 mW) nonlinear optic (NLO) exposed gel.

FIG. 8 is a graph of the increase in ratio of post to baseline elasticity of the treated gels comprised of a control group, a UVA exposed gel, a low power (100 mW) nonlinear optical (NLO) exposed gel and a high power (150 mW) nonlinear optical (NLO) exposed gel.

FIG. 9. (A), (B), and (C) illustrate three possible crosslinking patterns in the corneal tissue among an unlimited number of possibilities with the present invention.

FIG. 10 is a schematic representation of the optics of another embodiment of device.

FIG. 11 is a diagram of the effects in the corneal tissue of varying the beam diameter.

FIG. 12 is a diagram of the effects in the corneal tissue of varying the relative position of the focusing lens to the applanation cone or cornea.

FIG. 13 (A) An NLO CXL delivery device including a variable beam expander, xyz-galvo scanners to control CXL treatment geometry, and an objective. (B) Variable beam expander controls Rayleigh length of the laser focus thus allows for precise control of axial length of C×L region per pulse. (C) Z-galvo allows for precise control of depth of focus within the cornea.

FIG. 14. FEM model of a 2× change in mechanical stiffness of the central cornea shows that there would be up to 1.85 diopters of central corneal flattening depending on the diameter of the region. D=2.0 mm 0.75 D; D=3.0 mm 1.63 D; D=4.0 mm 1.85 D.

FIG. 15. Corneal collagen autofluorescence (CAF) induced by single 5 kHz, regeneratively amplified 780 nm femtosecond (FS) laser pulses at 2.4 μJ/pulse and an average power of 12 mW. (A) CAF image taken horizontal to the corneal surface showing effects of single pulses with spot separation of 3 μm. (B) CAF image taken vertical to the corneal surface showing corneal cross linking using 3 passes through the cornea at different depths.

FIG. 16. Femtosecond nonlinear optical parametric amplifier (NOPA). This system generates FS laser pulses at 760 nm and 20 kHz. The output from this device goes into the delivery system.

### DETAILED DESCRIPTION

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

It is known that collagen crosslinking can be caused using UV light and riboflavin in the cornea and that there is a correlation between collagen autofluorescence induced by crosslinking and the mechanical stiffening effects of UV-riboflavin. Autofluorescence is the natural emission of light by biological structures, such as mitochondria and lysosomes, when they have absorbed light, and is used to distinguish the light originating from artificially added fluorescent markers (fluorophores). We have established that collagen autofluorescence can be used to evaluate collagen crosslinking and that the intensity of autofluorescence is correlated with the amount of corneal stiffening. We have further developed preliminary data showing the NLO-PDT can induce increased corneal stromal autofluorescence in riboflavin soaked corneas. We also have data showing that NLO-PDT increases collagen gel stiffness, showing the proof of concept.

NLO-PDT uses very short pulsed laser light that can be accurately focused within tissues to activate photosensitizing chemicals such as riboflavin to generate free radicals within highly resolved spatial domains, axially and laterally. The very short-pulsed laser light used by NLO-PDT allows for precise focusing of high intensity light within very small volumes leading to nonlinear effects through multiple photon interactions. NLO-PDT allows for the use of lower energy laser light in the near-infrared region that has deeper depth of tissue penetration to activate photosensitizing chemicals that are normally activated by short wavelength, higher energy light that can cause cellular damage and has short depth penetration into tissues. Photosensitizers such as riboflavin that are excited by ultraviolet light (UV) are currently being used to treat corneal thinning by inducing oxygen radical generation leading to collagen crosslinking (CXL) and corneal stiffening. Additionally UV-CXL has been used as an anti-microbial method to treat corneal infections and to inhibit corneal swelling in bullous keratopathy.

A major drawback of UV-CXL is that there is no control over the volume of tissue where free radicals are generated when conventional UV light is used. This can lead to unwanted effects including cellular damage below the region of cross linking that may involve the corneal endothelium which is a nonregenerative cell layer in the cornea that is responsible for maintaining corneal transparency and limits the volume available for crosslinking. Therefore, the purpose of using NLO photoactivation is to generate free radicals only in the focal volume of the laser beam where NLO effects occur. This volume can be precisely controlled by lenses/objectives used to focus the light into the tissue, thereby leading to highly localized photoactivation.

NLO-PDT will allow for precise depth and area activation of photosensitizers that conventional UV-CXL lacks. Generation of free radicals by NLO femtosecond lasers can also be used to kill cells, bacteria, tumors and neovascular vessels growing into the avascular cornea with more precision then current approaches. The advantage of the disclosed NLO-PDT methodology is that activation of photosensitizer will occur only at the focal volume defined by the focusing objective of the laser. This will allow precise localization of oxygen radical generation and corneal crosslinking and anti-microbial and tumorcidal activity, as well as crosslinking in deeper corneal layers without damaging the corneal endothelium.

There are at least four immediate uses for localized NLO-PDT. First, collagen crosslinking and corneal stiffening can be used to more precisely stiffen weakened corneas, such as keratoconus and post-LASIK ectasia. Currently UV crosslinking is used clinically to treat these diseases. The disclosed approach will replace the current standard of care. Second, since crosslinking results in corneal stiffening and compensatory flattening and steepening in different regions, the disclosed NLO-PDT method can be used to precisely stiffen, flatten and steepen regions of the cornea to treat astigmatism and refractive errors associated with myopia, hyperopia and presbyopia. Third, the disclosed NLO-PDT methodology can be used to treat bacterial, fungal, and amoebic infections of the eye without antibiotics. Generation of free radicals is already used to sterilize tissue and fluids. NLO-PDT based oxygen radical generation can be used in a similar therapeutic modality with the disclosed methodology. Fourth, the disclosed NLO-PDT methodology can be used to kill labeled tumor cells in the eye following loading with photosensitizing dyes. The disclosed NLO-PDT methodology can be used to treat a range of clinical diseases including keratoconus, post-LASIK ectasia, astigmatism, myopia, hyperopia, infection and ocular tumors.

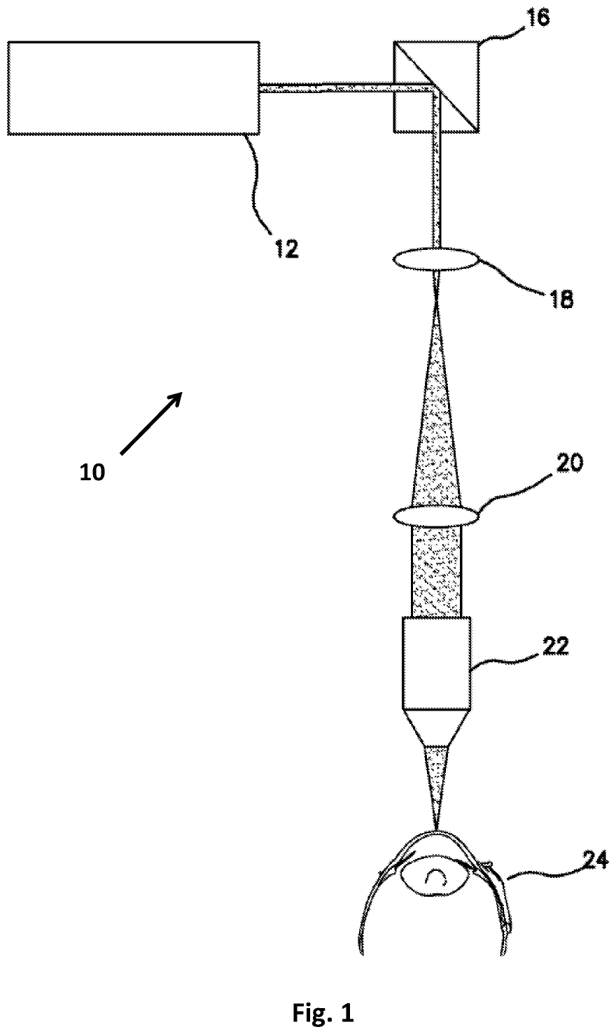

FIG. 1 is a simplified block diagram of an apparatus 10 implementing one embodiment of the invention. Femtosecond infrared pulsed laser 12 has a tunable output from 700 to 960 nm that is scanned by an x/y scan unit 16 through a beam expander (lenses 18 and 20) and focusing optics 22 into a cornea 24. For experimental purposes the focusing optics is a conventional objective able to selectively focus the pulsed light into a volume of 22 μm³ located at 5.5 mm below the objective tip. Depth and volume of focus can be selectively manipulated by modification and movement of the focusing optics 22.

Two-photon excited fluorescence (TPEF) occurs when a fluorophore absorbs two or more photons of near-infrared light (700 to 960 nm) and emits a visible light photon. Two-photon excited fluorescence differs from single photon excited fluorescence (SPEF) in that the two-photon excited fluorescence signal is generated only at the focal volume, is less phototoxic than single-photon excited fluorescence, exhibits dramatically improved axial resolution and has a deeper depth of tissue penetration.

In an experiment illustrating the disclosed embodiment, fresh enucleated rabbit eyes were treated with 0.1% riboflavin in a 20% dextran solution by volume for 30 minutes. The eyes were moved relative to the objective 22 using an x-y translational stage with lateral movement of 10 mm/sec with a 3 μm line separation. The central cornea region was exposed to 760 nm Chameleon femtosecond laser light at 190 mW using laser 12 and a 20× objective 22. The axial position of the beam focus was controlled by moving the eye relative to the fixed focal volume defined by objective 22. The corneas were then removed, fixed and evaluated for TPEF collagen autofluorescence, which was measured using a Zeiss multiphoton confocal microscope.

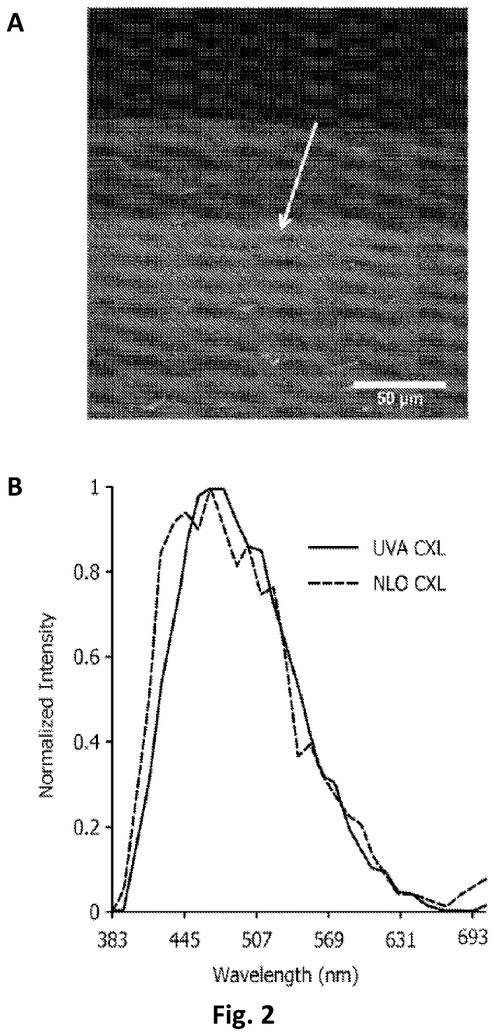

Multiphoton excitation of riboflavin within the corneal stroma generated fluorescence and free radicals leading to collagen crosslinking. NLO-PDCxl induced collagen autofluorescence within 9, 1 millimeter line scans with 3 micron line separation is shown in the TPEF image shown in FIG. 2a. The NLO-PDCxl autofluorescence spectrum is shown graphically in FIG. 2b and compared against UVA collagen crosslinking autofluorescence in the cornea after 30 minutes irradiation in FIG. 2b. The normalized collagen autofluorescence spectrum generated by NLO-PDCxl as shown in FIG. 2b is very similar to the autofluorescence spectrum generated by UVA crosslinking. Therefore, selectively focused femtosecond laser beams can be used to create collagen crosslinking and corneal stiffening with similar biological effects as are observed with the more uncontrolled UVA induced crosslinking of the prior art.

In another demonstration of the concept of the invention collagen hydrogels were made and their mechanical stiffening using the methodology of the invention was measured. Compressed collagen hydrogels were made by polymerizing 3 ml of rat-tail type-1 collagen gel (3 mg/ml) in a 24 well tissue culture plate. Gels were compressed to 100 micron thickness using conventional methods. To facilitate transport, gels were compressed onto #54 Whatman Filter discs having a central 7.6 mm diameter hole exposing the hydrogel for biomechanical testing and NLO CXL.

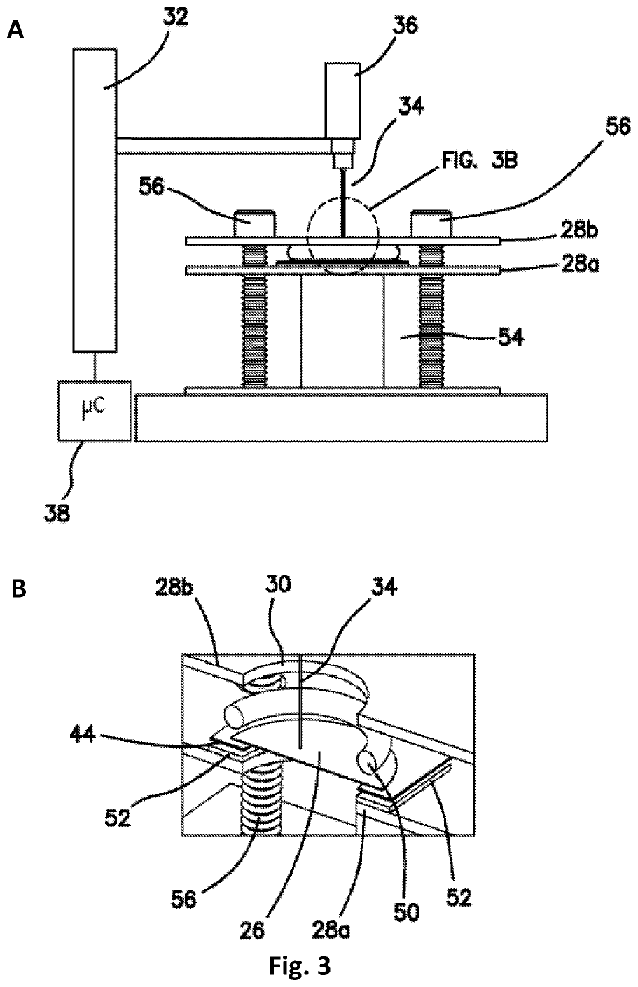

As shown in FIGS. 3a and 3b a jig was made to measure the elastic modulus of the gels 26, which were clamped between two steel plates 28a and 28b, each having a 7.6 mm diameter central hole 30 on a three dimensional control assembly 32. Plate 28a is mounted on a hollow transparent cylinder 54. Gel 26 is mounted on filter paper 44 and gasket 52 on top of plate 28a, each including a central hole 30 as best shown in FIG. 3b. An O-ring 50 is mounted on top of gel 26 followed by plate 38b. Plates 28a and 28b are bound together by compression from bolts 56. Gels 26 were then indented using a 250 μm diameter round tipped probe 34, as shown in FIG. 3b, attached to a force transducer 36 driven by automated electrical step motor within control assembly 32 controlled and recorded by computer 38 as shown in FIG. 3a. Each gel 26 was indented at the center through 1 mm depth at the velocity of 20 μm/sec and indenting force and depth recorded every 0.05 sec through 10 cycles. The elastic modulus. E, was then calculated using Equation 1, which is the modified Schwerin point-load solution of elastic modulus.

$$E = \frac{{( f ( v ) )}^{3} {a}^{2} P}{{δ}^{3} h}$$

$$f ( v ) \approx 1.049 - 0.146 v - 0.158 {v}^{2}$$

Where P is the indenting force, a the radius of hole 30, h the gel thickness, v the Poisson ratio, and δ the indenting depth.

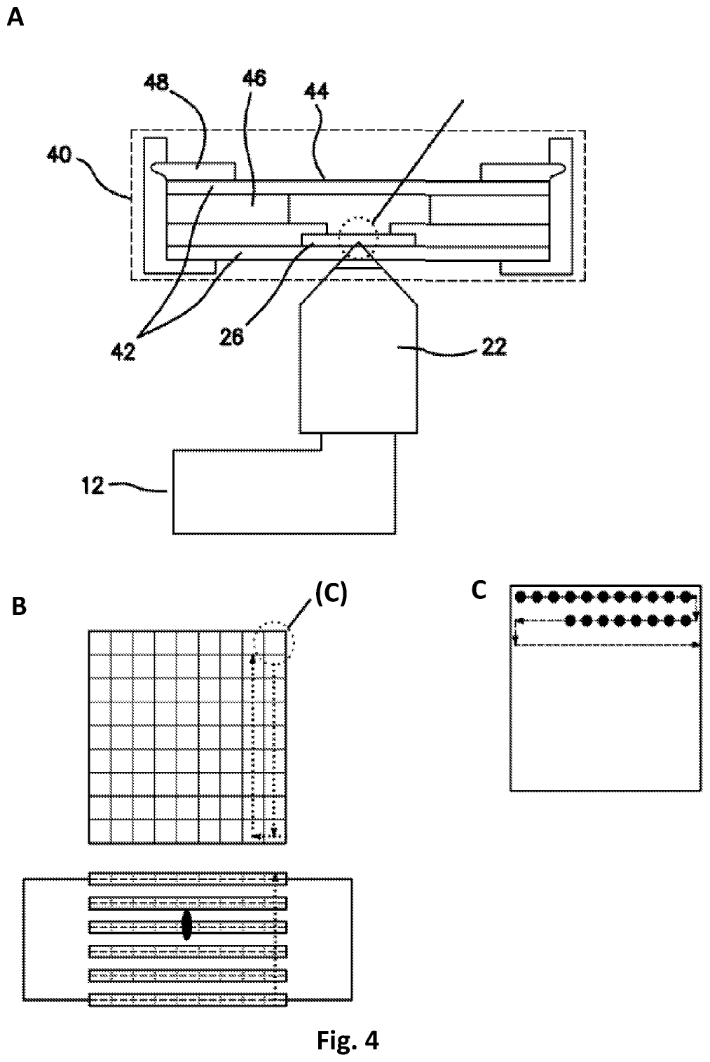

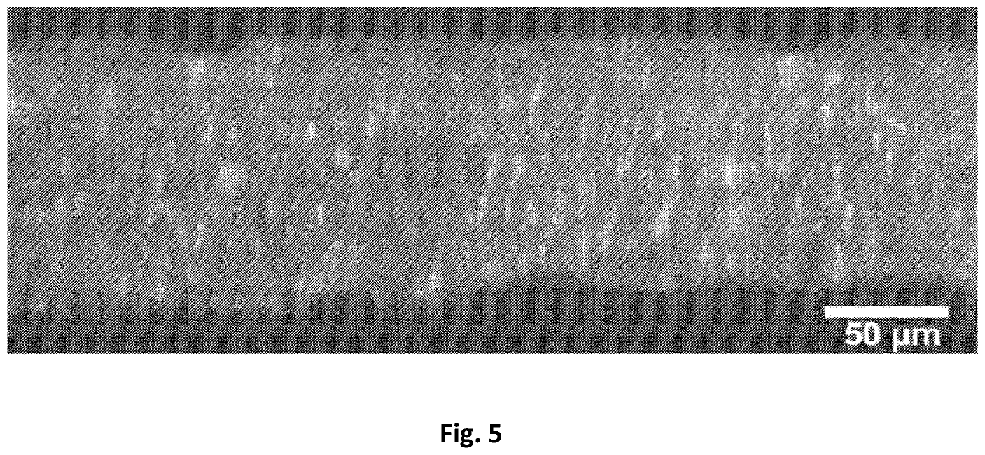

Gels 26 were then soaked in 0.1% riboflavin in phosphate buffered saline (PBS) and mounted in an NLO crosslinking chamber 40 as shown in FIG. 4a. The chambers 40 were then mounted onto a Zeiss 510 Meta confocal laser scanning microscope (CLSM) and gel thickness measured by second harmonic generation (SHG) imaging as shown in FIG. 5. NLO CXL was then performed by focusing a 100 mW (NLO I) or a 150 mW (NLO II), 760 nm femtosecond laser beam into the gel 26 using a 20× Zeiss apochromat objective lens 22 (NA=0.75). Gels 26 were scanned at 27.8 cm/sec velocity over a 5.2 mm×5.2 mm square area through the gel at 2 μm steps in a three dimensional tile scan as shown in FIGS. 4b and 4c. Control and UVA CXL gels 26 were left in the chamber 40 for the same duration as NLO CXL. For UVA CXL gels 26 were removed from the chamber 40 and exposed to 370 nm UVA light at 3 mW/cm² for 30 min over the same area as NLO CXL. The indenting force was then re-measured for each gel 26 as well as gel thickness.

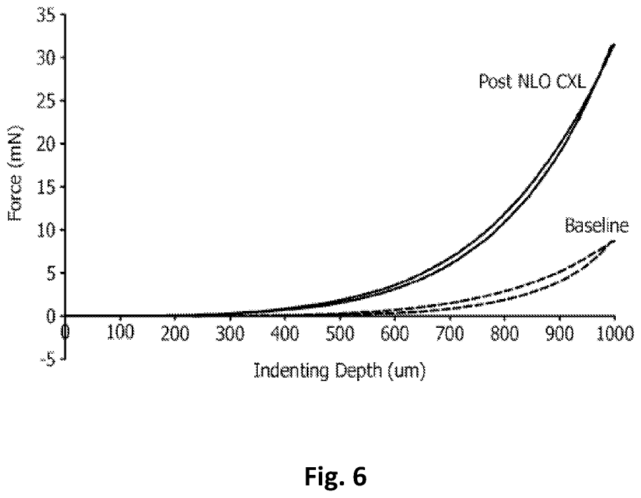

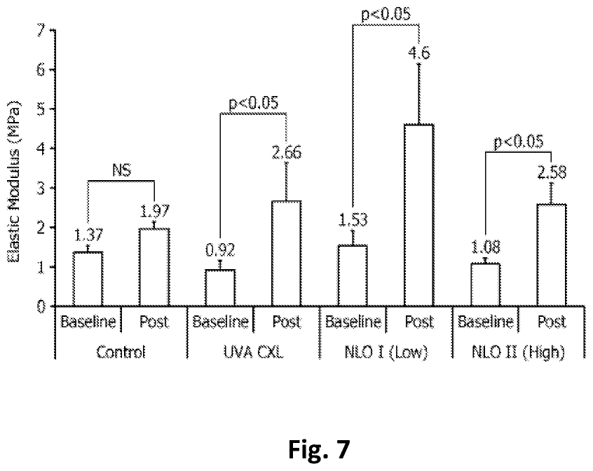

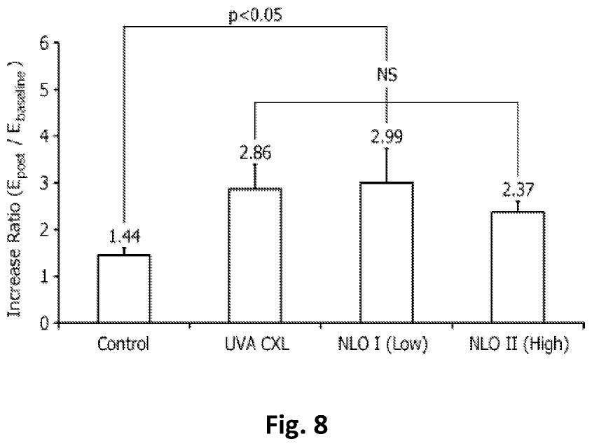

NLO collagen hydrogel crosslinking is shown in FIG. 6 at the 10^{th} cycle. NLO I treatment resulted in a marked increase in the indenting force suggesting that CXL and stiffening were induced by NLO I treatment. FIG. 7 shows baseline and post-treatment E values for each group before and after. Significantly increased post-treatment E values (p<0.05) were observed for all of CXL treatment groups. No significant difference was detected in the control group (p=0.22). Comparison of the ratio in E values between pre and post CXL (FIG. 8) showed no significant difference between UVA CXL and NLO CXL (p=0.38);

We thus show that nonlinear optical, multiphoton excitation of riboflavin using a femtosecond laser can induce collagen hydrogel crosslinking and mechanical stiffening similar to UVA CXL. Increased collagen autofluorescence in the cornea suggests that NLO CXL can stiffen the cornea. Because of the higher axial resolution of multiphoton processes, NLO CXL provides a safer and more effective therapeutic approach to treating corneal ectasia.

Ultraviolet A (UVA) mediated corneal crosslinking (UVA-CXL) is a known method to stiffen corneas, originally developed as a treatment for keratoconus (KC). Stiffening is achieved by using UV light to activate a photosensitizer such as riboflavin, which leads to the formation of free radicals that in turn causes the formation of additional crosslinks. Traditionally, the UV light is emitted by diodes used to effectively expose the entire cornea at one time.

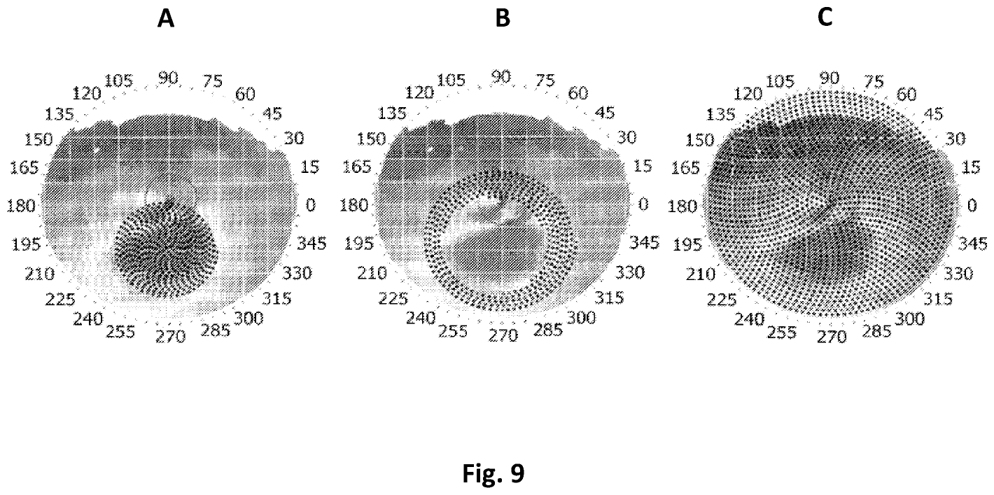

Two-photon corneal crosslinking (2P-CXL) uses an alternate approach to activate the photosensitizer. Here, ultrashort (femtosecond-range) infrared laser pulses are focused into the tissue. In the focal spot 25, which is typically only a few femtoliters in volume, two infrared photons interact to form a single UV photon, which then performs the photoactivation. This process is limited to a very small focal volume, and thus allows for very precise positional control of crosslinking. In addition to being able to crosslink only parts of the cornea as shown in FIG. 9a, it is possible to create almost any conceivable pattern as shown in the example of FIG. 9b. 2P-CXL further expands the capabilities of CXL by allowing crosslinking of the deeper layers of the cornea, which is not possible using the conventional approach. Using conventional UV diodes, only the anterior portion of the cornea can be crosslinked so as to avoid damaging the corneal endothelium, the deepest layer of the cornea. Without the endothelium, the cornea cannot function. Because the CXL volume is very limited in 2P-CXL, crosslinking can be performed close to the endothelium without risking damage.

However, the small focal volume is also the main drawback of 2P-CXL. Since only a small portion of the cornea is being crosslinked at a time, two photon crosslinking is a process which is very slow. Conventional UVA-CXL has an exposure time of 30 minutes. Research is currently ongoing to reduce that time to 10 minutes or less. By contrast, using a small, micron-sized focal volume as contemplated here, crosslinking a similar corneal volume would take up to 8 hours. This is dearly beyond a reasonable clinical time span during which it can be practically used as a therapeutic method. It is preferable that therapeutic procedures be completed within short patient exposure times of the order of tens of minutes or less than 10 minutes in order for the treatment duration to be clinically accepted. In the preferred embodiment a clinical exposure of cross-linking the entire cornea is approximately 5 minutes or less in duration is the acceptable clinically accepted time.

To address this problem, there are two possible approaches:

a. Increase the scanning speed by moving the focal spot more rapidly across the cornea. While feasible from a mechanical standpoint, it would also require significantly higher energies in order to activate the photosensitizer. To achieve measurable crosslinking, power levels that far exceed the FDA-allowed limits would have to be employed. A safe intensity of the laser light is understood to be equal to or less than the FDA maximum allowed limit for laser exposures, which may be dependent on the kind of tissue irradiated and the wavelength of the light. Currently, the FDA has set a safe maximum limit on femtosecond lasers of 46.1 mWatts of delivered power. It must be understood that the safe maximum limit may be varied by the FDA over time and may depend on the nature or modulation of the laser and pulse or irradiation delivered. A variation of this proposed approach is illustrated by Lubatschowski's multifocal approach disclosed in US Patent Pub. US 2007/0123845, which proposes splitting up the beam and using more than one focal spot simultaneously. Setting the engineering obstacles to this approach aside, because the beam is split into several spots, the unsplit original beam would have to be several times more powerful than the safe intensity. The resulting power levels of the originating beam would be markedly higher than allowed by FDA safety regulations.

b. Expand the focal spot size, thereby crosslinking larger volumes at the same time so that the selected volume of the tissue to be treated can be scanned more quickly. Essentially, this is a hybrid approach sacrificing some positional accuracy for much higher scanning speeds.

The disclosed device uses a single, low numerical aperture (NA) lens. The lower the NA, the larger the focal volume. The NA of a lens is dependent on its focal length, which is a fixed parameter, and on the diameter of the incoming beam. Essentially, in order for the lens to achieve its maximum possible NA and therefore its smallest focal spot size, the beam has to completely fill or even overfill the back aperture of the lens. The beam diameter is inversely proportional to the focal volume with all other parameters kept constant. By making the beam diameter smaller than the lens diameter, the lens becomes “less effective”. Therefore, by varying the diameter of the laser beam, we can vary the effective NA of the lens, and thereby vary the focal spot volume.

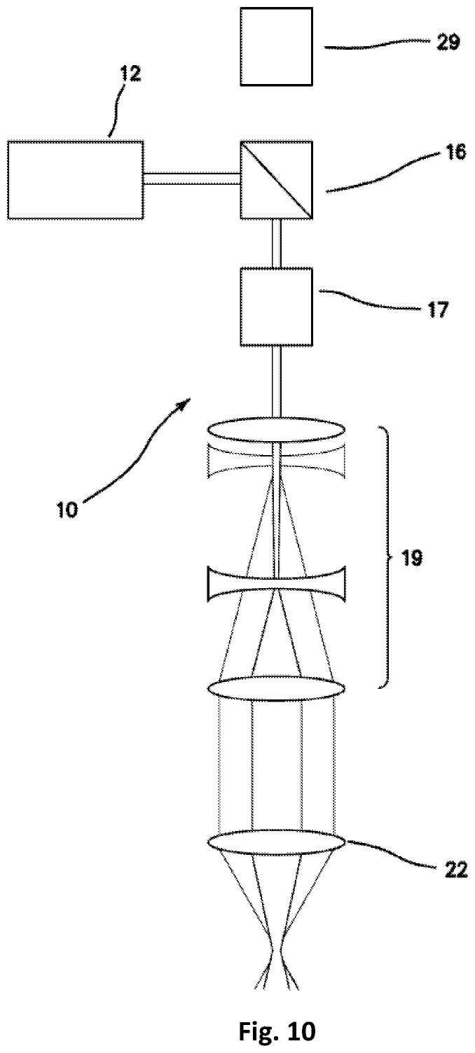

FIG. 10 is a schematic representation of the optics of another embodiment of device 10. Infrared laser pulses are generated by the femtosecond laser 12 and sent through a dichroic beam splitter 16. The beam splitter “sorts” light by wavelength in that it reflects certain wavelengths, in this case infrared light, while letting others pass through. Being infrared, the laser beam is reflected into the X/Y scan unit 17. This unit is comprised of two or more computer-controlled mirrors that can move the beam in x and y directions or in a plane perpendicular to the depiction of FIG. 10. The scanned beam then enters a variable beam expander 19. Essentially a variable-zoom telescope, this computer-controlled expander 19 allows us to adjust the beam diameter. The adjusted beam is then focused into the tissue by a focal or focusing lens 22, the effective NA of which is controlled by the beam diameter. In the illustrated embodiment part of multiphoton UV light created in the focal spot is relayed back through the optical system and, due to its lower wavelength, passes through the beam splitter 16 into a spectral analyzer 29 which is used to monitor the procedure. A clinical embodiment of the device 10 might include the analyzer 29 as an option.

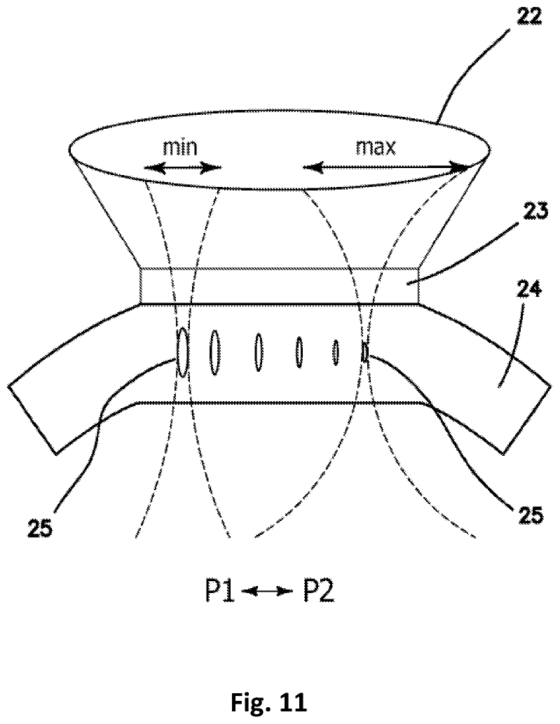

The effects of varying the beam diameter are shown in FIG. 11. To ensure a smooth, even optical surface of the cornea 24, a single-use applanation cone 23 is used to applanate or flatten the central cornea 24 and to optically couple the patient's eye to device 10. At its minimum setting, the beam has a diameter significantly smaller than that of the focusing lens 22, resulting in a large focal volume P1 shown in the left of FIG. 11. By increasing the diameter, the focal volume is decreased, until the beam diameter is greater than the diameter of the focusing lens, allowing the lens to act at maximum efficiency and resulting in a very small focal volume P2 shown in the right of FIG. 11 as a comparative example. We can therefore choose between speed and precision as necessary. The larger the focal volume, the faster a selected volume of the cornea 24 can be scanned. Conversely, the smaller the focal volume the slower a selected volume of the cornea 24 can be scanned. Scanning speed and focal volume are selected to achieve clinically acceptable exposure times of a selected volume of cornea 24 using a pulsed laser light at safe intensities to effectively activate the photosensitizer. The correct selection of parameters can be determined empirically in each case or by calculation using first principles of the photomediation of tissue.

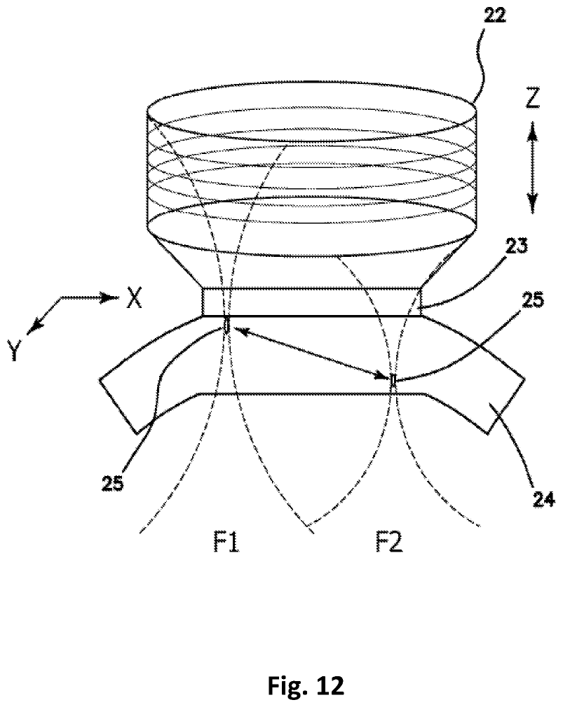

In the depiction of FIG. 12, the z-direction is vertical on the plane of the drawings, the x-direction is to the left in the plane of the drawing and the y direction is perpendicular to the plane of the drawing. The focal spot 25 can be precisely positioned and moved in three dimensions. Its x, y position of the focal spot 25 relative to applanation cone 23 and hence cornea 24 is controlled by the x/y scan unit 17. To control its z position in the tissue or depth in the tissue, the focusing lens 22 is moved in the z-direction relative to the applanation cone 23 and thus relative to the cornea tissue 24 between the position shown in the left of FIG. 12 as F1 and on the right of FIG. 12 as F2. In the diagram of FIG. 12, focusing lens 22 is shown in multiple positions, with the resulting location of the focal spot 25 in corneal tissue 24 being shown only in the two extremums of the corneal positions corresponding to the extremum positions of focusing lens 22. Any vertical position between the corneal extremums can be chosen by positioning lens 22 in a corresponding relative z-displacement with respect to the applanation cone 23. The z-displacement of lens 22 is coordinated by computer with the x, y scanning of scan unit 17 to provide the desired coverage of the selected volume of the tissue. Thus, not only is the absolute magnitude of the volume selected, but also its three dimensional location in the tissue is selected.

The three dimensional movement of a variable volume focal spot 25 allows us to create almost arbitrary crosslinking patterns in the tissue with clinically acceptable exposure times and safe levels of laser exposure. FIGS. 9a-9c show examples of possible patterns mapped onto a surface topography map of a keratoconus cornea 24. In addition to following the conventional protocol for KC crosslinking by exposing the entire cornea as shown in FIG. 9c, we can limit crosslinking to just the cone area as shown in FIG. 9a or create a stabilizing annulus by crosslinking the area around the cone as shown in FIG. 9b.

Lubatschowski's device uses a 0.3 NA lens, which gives a theoretical two photon volume of less than 19 femtoliters. The variable or effective NA methodology and apparatus disclosed here allows us to vary the NA below 0.3 to between 0.16 and 0.08 with corresponding focal volumes between 150 and 2500 femtoliters. At its maximum setting, this gives a focal volume 130 times greater than that of the 0.3 NA lens. To achieve similar speeds, a multifocal method and apparatus as disclosed by Lubatschowski with a 0.3 NA lens would have to provide an array of at least 11 by 11 or 121 separate spots of laser light to achieve the same effect with a corresponding increase of intensity of the originating or unsplit laser beam. The NA values of 0.16/0.08 and the corresponding focal volumes disclosed above are based on the illustrated embodiment. However it must be understood that these values are by no means the absolute theoretical limits of a variable NA beam delivery system according to the present scope of the invention. By using a different focal lens 22 with a larger diameter and different focal length, for example, it is possible to increase the range of focal volumes further consistent with the teachings and scope of the invention.

### Regenerative Amplifier Technology

Short optical pulses are typically generated by mode locked oscillators with pulse energies between a few nanojoules and tens of microjoules. To reach significantly higher pulse energies, an amplifier or amplifier chains may be employed.

A regenerative amplifier is a device that is used for strong amplification of individual pulses from a train of low-energy pulses emitted by a laser oscillator, usually with ultrashort pulse durations in the picosecond or femtosecond domain. Multiple passes through a gain medium (nearly always a solid-state medium) are achieved by placing the gain medium in an optical resonator, together with an optical switch, usually realized with an electro-optic modulator and a polarizer. As the number of round trips in the resonator can be controlled with the optical switch, it can be very large, so that a very high overall amplification factor (gain) is achieved.

### Femtosecond Laser Parameters

Near-infrared optical pulses may be generated within a range of about 700 nm to 2500 nm. In some embodiments, the optical pulses have a wavelength of from 700-750 nm, 750-800 nm, 800-850 nm, 850-900 nm, 900-950 nm, 950-100 nm, 1000-1050 nm, 1050-1100 nm, 1100-1150 nm, 1150-1200 nm, 1200-1250 nm, 1250-1300 nm, 1300-1350 nm, 1350-1400 nm, 1400-1450 nm, 1450-1500 nm, 1500-1550 nm, 1600-1650 nm, 1650-1700 nm, 1700-1750 nm, 1750-1800 nm, 1800-1850 nm, 1850-1900 nm, 1900-1950 nm, 1950-2000 nm, 2000-2050 nm, 2050-2100 nm, 2100-2150 nm, 2150-2200 nm, 2200-2250 nm, 2250-2300 nm, 2300-2350 nm, 2350-2400 nm, 2400-2450 nm, or 2450-2500 nm.

The FS laser pulses amplified by a nonlinear optical parametric amplifier (NOPA) may have frequencies of from about 0.5 to about 100 kHz. In some embodiments, the frequency is from about 1-5 kHz, 5-10 kHz, 10-15 kHz, 15-20 kHz, 20-25 kHz, 25-30 kHz, 30-35 kHz, 35-40 kHz, 40-45 kHz, 45-50 kHz, 50-55 kHz, 55-60 kHz, 60-65 kHz, 65-70 kHz, 70-75 kHz, 75-80 kHz, 80-85 kHz, 85-90 kHz, 90-95 kHz or 95-100 kHz.

Amplified femtosecond laser pulses provide higher pulse energy ranging from 0.1-100 μJ. In some embodiments the pulse energy is from 0.1-1 μJ, 1-2 μJ, 1-3 μJ, 2-3 μJ, 3-4 μJ, 4-5 μJ, 5-6 μJ, 6-7 μJ, 7-8 μJ, 8-9 μJ, 9-10 μJ, 10-20 μJ, 20-30 μJ, 30-40 μJ, 40-50 μJ, 50-60 μJ, 60-70 μJ, 70-80 μJ, 80-90 μJ or 90-100 μJ. This is different compared to Lubatschowski's approach, as disclosed in US Application Publication No. 2007/0123845, which teaches pulse energies ranging from 0.1 to 100 nJ.

In some embodiments, the collagen cross linking increases the material stiffness of the tissue (e.g., a cornea) by 0.1- to 10-fold. The increase in material stiffness may be 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, or 1000%.

By varying the area of tissue cross linking and/or by cross linking within different geometries, a tissue can be modified to produce a desired effect. For example, the area of cross linking may be varied between 0.1 to 10 mm, or over ranges of from 1-2 mm, 2 to 4 mm, 4-6 mm, 6-8 mm or 8-10 mm. A variable beam expander allows control of the volume of cross linking. Using different geometries a graded flattening of the cornea can be obtained. For example, the cross linking can be done within a volume having the following non-limiting shapes of a lens, a sphere, an ellipse, a donut shape, a cylinder shape or an oblong shape having at least one diameter of from 0.1 to 10 mm, including diameter ranges from 1-2 mm, 2 to 4 mm, 4-6 mm, 6-8 mm or 8-10 mm.

The methods described herein also have an energy density below 100 J/cm², compared to the Lubatschowski approach, which has an energy density above 100 J/cm².

### Example 1

### NLO CXL Using Single Pulse, Amplified 760 nm Femtosecond Laser

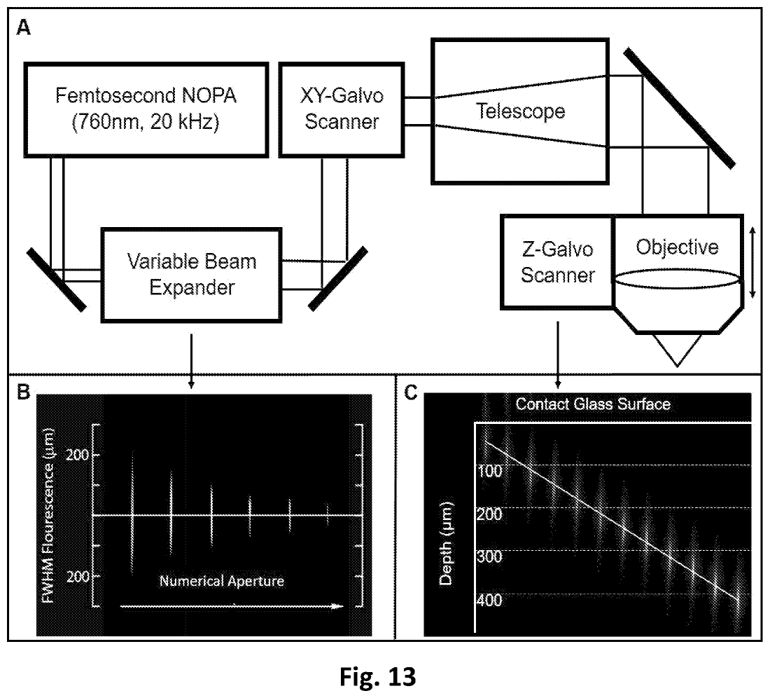

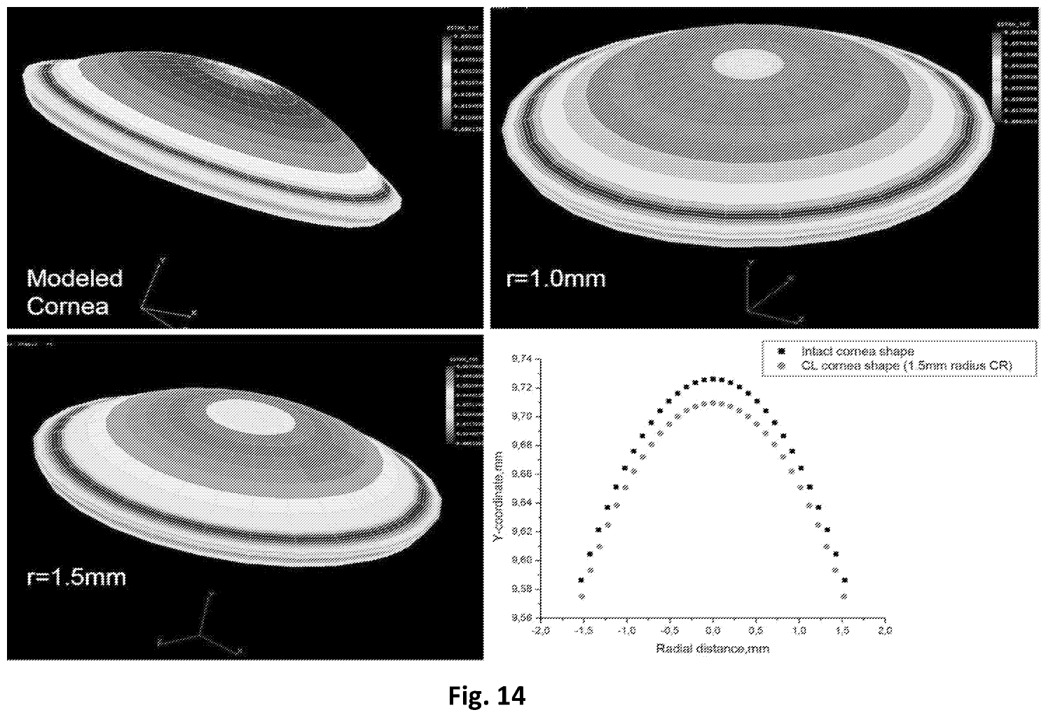

We have built an FS laser beam delivery device for NLO CXL using FS lasers (FIG. 13). Using this device, we have established that NLO CXL can lead to significant mechanical stiffening within the cornea and have demonstrated that the area of CXL can be precisely controlled by the delivery device to create different geometries (Bradford. S. M. et al. 2017 Biomed Opt Express 8(10): 4788-4797). We have also used finite element modeling (FEM, see, e.g., Freutel et al., 2014 Clin Biomech (Bristol, Avon) 29(4): 363-372, and Zhong and Smith, 2016 J Appl Mech Eng 5(6): 1-5) to predict the effect of corneal CXL on corneal shape and refractive power. FEM is a mathematical modeling algorithm that can solve problems in engineering and mathematical physics to assess the effects of stress and strain on structures based on their measured material properties. Assuming that CXL increases the material stiffness of the cornea by two fold, FEM modeling can be used to predict the effects of different CXL geometries on corneal shape and hence refractive power. While many different geometries (cylinders, torus, lens) and placement (central, paracentral, anterior, posterior) may be considered, in our first analysis using FEM models we modeled the effects of varying the diameter of a CXL cylinder of 2 to 4 mm diameter on corneal shape (FIG. 14). The results indicate that using cylinders extending from the anterior to posterior cornea of different diameters we can induce a graded flattening of the cornea from 0.75D (2 mm diameter) to 1.85D (4 mm diameter) providing a basis for correction of mild degrees of myopia. While this method using a 76 MHz FS with 10.5 nJ per pulse achieved rapid, localized, and spatially controllable CXL, the required laser power of 800 mW is 20 fold higher than the American National Standards Institute (ANSI) limit (46.1 mW) for use in humans. The purpose of this study was to determine whether a single, regeneratively amplified 760 nm FS laser pulses at 5 kHz with ˜2 μJ pulse energy, a 500 fold higher pulse energy than used with the 76 MHz FS laser, could be used to photoactivate riboflavin within the cornea and induce CAF equivalent to that achieved with the 75,600 pulses using the FS oscillator. Single pulse photoactivation would substantially reduce the total power required for CXL from 800 mW to 12 mW and be below the ANSI limits.

Methods: The same variable numerical aperture (NA), custom laser scanning delivery system with adjustable focal depth was used as in our previous studies, 800 nm FS pulses from a regenerative amplifier (5 kHz) were tuned to 1520 nm in an optical parametric amplifier (Coherent Inc. Santa Clara, Ca). The 1520 nm laser pulses were then frequency doubled in a custom bismuth triborate (BiB₃O₆) nonlinear crystal (Newlight Photonics, Ontario Canada) to 760 nm and then aligned into our delivery system. Rabbit corneas soaked in 0.5% Riboflavin/PBS with dextran (20%) were raster scanned with 0.1 NA, 5 mm/s scan velocity, and 12 mW of average power. CAF was used to detect corneal collagen CXL.

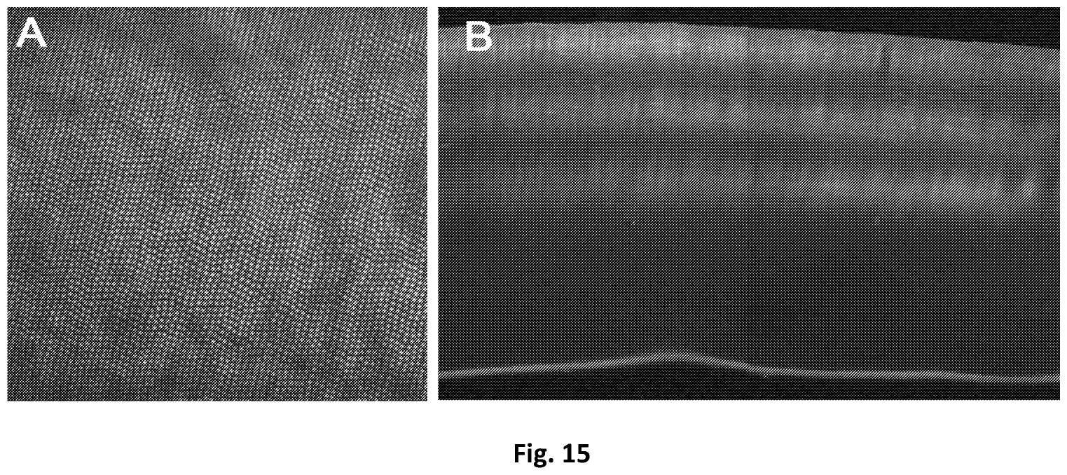

Results: We have shown that a single amplified FS pulse can generate CAF within rabbit corneas (Mikula et al. 2017 “Precise corneal crosslinking (CXL) using a 5 KHz amplified femtosecond laser,” presented at The Association for Research in Vision and Ophthalmology annual meeting, Baltimore, Md.). Representative CAF images are presented in FIG. 15. As shown in FIG. 15A, when the cornea is cut parallel to the surface, each regeneratively amplified FS laser pulse of approximately 150 FS duration leads to a single, isolated spot of 3 μm in the cornea that shows CAF. As depicted there are a series of dots, each representing a single FS laser pulse as the bean is scanned at 5 mm/sec over the corneal surface. When the cornea is cut in cross section as shown in FIG. 15B, each spot can be identified in the section as representing a larger, cylindrical volume that has the same width as the spot by an extended length 173±14 μm. Also in FIG. 15B, different depth within the cornea show CAF, representing different depths of focus of the delivery device resulting in multiple regions of CXL.

Conclusion: Using this approach we have established that a single, regeneratively amplified. FS laser pulse from a 5 kHz FS laser providing 2.4 μJ pulse energy at 12 mW average power can be used to induce collagen CXL within the cornea. The increase in pulse energy, using a 5 KHz FS laser, allows for a dramatic decrease in the overall power, satisfying ANSI limits and getting rid of the need for overlapped pulses (Table 1). Also when a single amplified pulse is used, instead of applying overlapping, multiple pulses per spot of tissue, the volume can be scanned much faster using higher repetition rate lasers (5-50 KHz), thereby reducing overall procedure time.

TABLE 1

| | | 76 MHz | 5 KHz Amplified |
|---|---|---|---|
| Light Parameters | UVA-CXL | NLO CXL | NLO CXL |
| Pulse Energy | NA | 10.5 nJ | 2.4 μJ |
| Pulse # (3 μm area) | Continuous | 75,600 pulses | 1 pulse |
| Treatment time | NA | 600 ms | 100 fs |
| Average Power | 3 mW | 800 mW | <12 mW |
| Total Energy | 5.4 J | 480 J | 7.2 J |

These results provide a basis for using a larger single pulse energy (e.g., 100, 200, 400, 500 times larger than the pulse energy used in the 76 MHz NLO CXL), while still respecting ANSI limits.

### Example 2

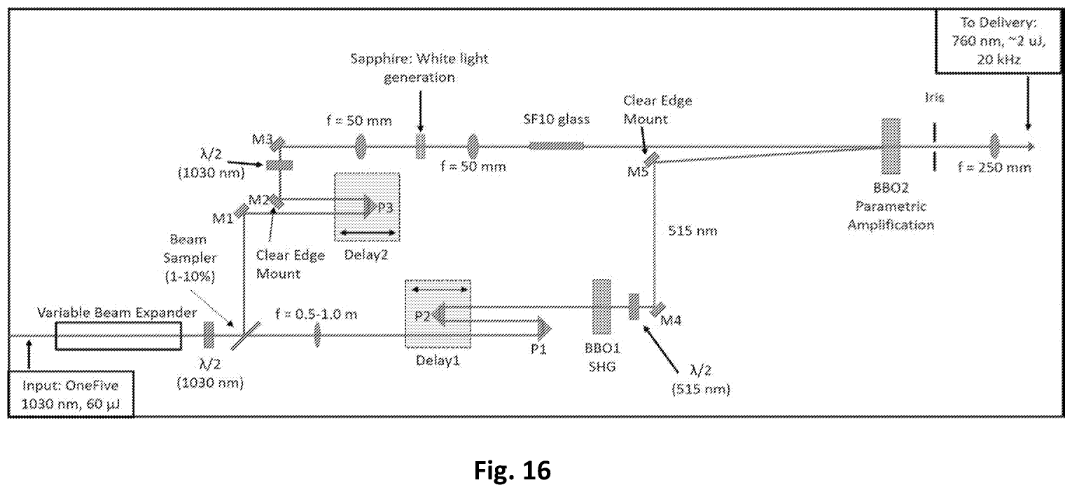

We have designed a Nonlinear Optical Parametric Amplifier, NOPA (FIG. 16), to deliver high energy, single pulse FS laser light to the NLO CXL delivery device. This system uses a regeneratively amplified FS laser that provides 1030 nm, 60 uJ FS laser pulses at 5-50 kHz. The light is split into two pathways within NOPA producing a white light seed beam and an amplifier beam (FIG. 16). The white light seed beam is directed into a sapphire crystal generating a broad white light spectrum. The amplifier beam is frequency doubled in the first BBO crystal and then subsequently overlapped spatially and temporally with the wavelength of interest from the white light beam. The two beams overlap spatially and temporally within the second BBO, thus resulting in parametric amplification of the wavelength of interest, namely 760 nm. Using our novel device, which includes the regeneratively amplified FS laser, NOPA and the NLO CXL delivery device, uniquely permits irradiation of a target, using regenerative amplification of FS laser pulses from 5-50 kHz, and provides pulse energies of from 0.1-100 μJ pulse energy at <46.1 mW average power.

It is understood that the examples and embodiments described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the embodiments.

What is claimed is:

1. A laser beam delivery system comprising an amplified femtosecond (FS) laser device coupled to a nonlinear optical parametric amplifier (NOPA) configured to select a FS laser wavelength and to amplify input amplified femtosecond (FS) laser pulses of from 700 to 2500 nm to generate a single, parametrically amplified output FS pulse having a pulse energy of from 0.1-100 μJ, wherein the NOPA uses an average power of below 46.1 mW to amplify the input FS laser pulses.

2. The laser beam delivery system according to claim 1, wherein the amplified FS laser device is configured to provide a repetition rate of 5 kHz to 50 kHz pulses.

3. The laser beam delivery system according to claim 1, wherein the NOPA is configured to parametrically amplify 760 nm pulses.

4. The laser beam delivery system according to claim 1, wherein the system is configured to focus 760 nm light with a variable 0.1-0.3 numerical aperture (NA) objective.

5. The laser beam delivery system according to claim 1, wherein the NOPA is configured to provide a single output pulse of about 2 μJ pulse energy having an average power of about 12 mW or less.

6. The laser beam delivery system according to claim 1, wherein the system comprises a tracker that automatically monitors position of a subject or a tissue so that the device is able to compensate for movement of the subject or tissue.

7. A method of nonlinear optical photodynamic irradiation of a target, the method comprising exposing the target to a single amplified femtosecond laser pulse, wherein the amplified femtosecond laser pulse has a wavelength of from about 700 nm to 2500 nm, and wherein the single amplified femtosecond laser pulse has a pulse energy of from 0.1-100 μJ and an average power of less than 46.1 mW.

8. The method according to claim 7, wherein the single amplified femtosecond laser pulse is applied at an energy density below 100 J/cm².

9. The method according to claim 7, wherein the single pulse is for a duration of about 150 femtoseconds.

10. The method according to claim 7 comprising using regeneratively amplified pulses from 5 kHz to 50 kHz.

11. The method according to claim 7 comprising using pulse energies of between 0.1-100 μJ.

12. The method according to claim 7 comprising using energy densities of 1-100 J/cm2.

13. The method according to claim 7, further comprising pretreating the target with a photosensitive agent which is capable of generating free radicals within the treatment volume upon irradiation.

14. The method of claim 7, wherein the photosensitive agent comprises riboflavin.

15. A method of nonlinear optical photodynamic therapy of a tissue, the method comprising exposing the tissue to a single amplified femtosecond laser pulse, wherein the amplified femtosecond laser pulse has a wavelength of from about 700 nm to 2500 nm to minimize cellular damage by reducing energy level of the laser light and increasing its depth of penetration into the tissue, wherein the single amplified femtosecond laser pulse has a pulse energy of from 0.1-100 μJ and an average power of less than 46.1 mW.

16. The method according to claim 15, wherein the single amplified femtosecond laser pulse is applied at an energy density below 100 J/cm².

17. The method according to claim 15, wherein the single pulse is for a duration of about 150 femtoseconds.

18. The method according to claim 15 comprising using regeneratively amplified pulses from 5 kHz to 50 kHz.

19. The method according to claim 15 comprising using pulse energies of between 0.1-100 μJ.

20. The method according to claim 15 comprising using energy densities of 1-100 J/cm2.

21. The method according to claim 15, wherein the tissue is a cornea.

22. The method according to claim 21, comprising applying specific geometric patterns of collagen crosslinking (CXL) to induce defined and controllable corneal stiffening.

23. The method according to claim 22, producing 2 diopters or less of corneal flattening and/or steepening.

24. The method according to claim 22, wherein refractive correction of low degrees of myopia, hyperopia, presbyopia and astigmatism is achieved.

25. The method according to claim 15, further comprising pretreating the tissue with a photosensitive agent which is capable of generating free radicals within the treatment volume upon irradiation.

26. The method of claim 15, wherein the photosensitive agent comprises riboflavin.

27. The method of claim 15, wherein the pulsed infrared laser light within the tissue provides sufficient intensity and length of irradiation to cause collagen crosslinking (CXL).

28. The method of claim 15, wherein the pulsed infrared laser light within the tissue provides sufficient intensity and length of irradiation to effectively provide anti-microbial mediation.
